(19) United States
(12) Patent Application Publication
(10) Pub. No.: US 2008/0241211 A1
(43) Pub. Date: Oct. 2, 2008

(54) DEVICE WHICH ENHANCES THE BIOLOGICAL ACTIVITY OF LOCALLY APPLIED GROWTH FACTORS WITH PARTICULAR EMPHASIS ON THOSE USED FOR BONE REPAIR

(75) Inventors: Bo Han, Temple City, CA (US); Marcel Nimni, Santa Monica, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/057,236

(22) Filed: Mar. 27, 2008

Related U.S. Application Data
(60) Provisional application No. 60/908,262, filed on Mar. 27, 2007.

(51) Int. Cl. A61K 9/00; A61K 38/16; A61K 31/352; A61P 19/08

(52) U.S. Cl. 424/423; 424/484; 514/12; 514/456

(57) ABSTRACT

This invention provides a novel medical appliance for repairing, regenerating, maintaining, and/or augmenting a bone. The medical appliance generally includes an osteoinductive agent, an osteoinductive enhancer, and a carrier matrix. Also disclosed are methods, compositions, kits, and bone matrix formulations for regenerating, maintaining, and/or augmenting a bone. Exemplary preferred osteoinductive agents include growth factors such as BMP and TGF-β. Exemplary preferred osteoinductive enhancers include phytoestrogens such as naringin.

### CROSS-REFERENCE TO RELATED APPLICATIONS

This application claims an invention which was disclosed in Provisional Application No. 60/908,262, filed Mar. 27, 2007, entitled “DEVICE WHICH ENHANCES THE BIOLOGICAL ACTIVITY OF LOCALLY APPLIED GROWTH FACTORS WITH PARTICULAR EMPHASIS ON THOSE USED FOR BONE REPAIR”. The benefit under 35 USC §119(e) of the United States provisional application is hereby claimed. The above priority application is hereby incorporated herein by reference.

### FIELD OF THE INVENTION

The present invention relates generally to the field of bone repair, regeneration, maintenance and augmentation. More particularly, the present invention relates to a device which contains an osteoinductive agents, and an osteoinductive enhancer that can be used at the site of fracture repair or of bone healing to enhance osteogenesis. The present invention also relates to methods, compositions, bone matrix formulations for bone repair, regeneration, maintenance, and augmentation.

### BACKGROUND OF THE INVENTION

Repair of bone lesions, enhancement of bone formation around implants, and procedures that require significant amounts of new bone formation, such as spinal fusion, present important clinical challenges in medicine. For such applications, autologous cancellous bone (“ACB”) is considered the gold standard for bone grafts.

ABC is formed by the trabecular bone which is porous and highly cellular. It stimulates the bone formation because it provides live cells and growth factors. ACB is osteoconductive, is non-immunogenic, and, by definition, has all of the appropriate structural and functional characteristics appropriate for the particular recipient (it is taken from the recipient's own body). Unfortunately, ACB is only available in a limited number of circumstances. Some individuals lack ACB of appropriate dimensions and quality for transplantation. Moreover, donor site morbidity can pose serious problems for patients and their physicians.

In view of the limited applicability of ACB, much research has been focused on the identification or development of alternative bone graft materials. Towards this end, demineralized bone matrix (“DBM”) implants have been reported to be particularly useful (see, for example, U.S. Pat. Nos. 4,394,370; 4,440,750; 4,485,097; 4,678,470; and 4,743,259; Mulliken et al., Calcif Tissue Int. 33:71, 1981; Neigel et al., Opthal. Plast. Reconstr. Surg. 12:108, 1996; Whiteman et al., J. Hand. Surg. 18B:487, 1993; Xiaobo et al., Clin. Orthop. 293:360, 1993; each of which is incorporated herein by reference).

Demineralized bone matrix is typically derived from cadavers. The bone is removed aseptically and/or treated to kill any infectious agents. The bone is then particulated by milling or grinding and then the mineral component is extracted (e.g., by soaking the bone in an acidic solution). The remaining matrix is malleable and can be further processed and/or formed and shaped for implantation into a particular site in the recipient. Demineralized bone prepared in this manner contains a variety of components including proteins, glycoproteins, growth factors, and proteoglycans. Following implantation, the presence of DBM induces cellular recruitment to the site of implantation. The recruited cells may eventually differentiate into bone forming cells. Such recruitment of cells leads to an increase in the rate of wound healing and, therefore, to faster recovery for the patient. However, the osteoinductive abilities of commercially available DBM formulations are highly variable (FIG. 1). It has been observed that the osteoinductive ability of a DBM formulation is in proportion to the respective formulation's DBM content. This osteoinductivity-DBM content dependency sets a limit on the range and versatility of DBM formulations, since for every portion of an insert carrier that is added, an essentially linear proportional trade-off in the osteoinductivity per weight must be sacrificed. It stands to reason that if the active ingredients of DBM (i.e., the growth factors) may be extracted or synthesized, more versatile and yet equally active grafting materials may be created.

Over the last few years, growth factors, either natural or synthetic, have been finding significant applications in this connection. Unfortunately, delivery of such growth factors to the site of repair continues to be a unsolved technical challenge. Such growth factors, which belong to a family known as TGF-β, and which include BMP's (bone morphogenetic proteins) are widely used. Being proteins, they are subjected to biodegradation and loss of activity.

In some instances, various scaffolds have also been used to deliver such growth factors to the site of injury, collagen fibers being the most widely employed.

Overall, current bone and cartilage graft formulations have various drawbacks. First, while the structures of most bone or cartilage matrices are relatively stable, the active factors within the matrices are rapidly degraded. The biologic activity of the matrix implants may be significantly degraded within 6-24 hours after implantation, and in most instances matrices are believed to be fully inactivated by about 8 days. Therefore, the factors associated with the matrix are only available to recruit cells to the site of injury for a short time after implantation. For much of the healing process, which may take weeks to months, the implanted material may provide little or no assistance in recruiting cells.

Searches for novel forms of delivery and ways to stabilize and modulate the biodegradation of such delivery matrices are ongoing.

### SUMMARY OF THE INVENTION

In view of the above, it is an object of the present invention to provide a new approach to bone matrix formulation that does not suffer from the proportional osteoinductivity limitation.

It is a further object of the present invention to provide new methods, tools, devices, and compositions to improve the art of bone repair, regeneration, maintenance, and augmentation.

There and other objects of the present invention, which will become more apparent in conjunction with the following detailed description of the preferred embodiments, either along or in combinations thereof, have been satisfied by the discovery of an osteoinductive enhancer which is capable of enhancing, or amplifying the biological activities of osteoinductive agents such as BMP and TGF-β.

In a first aspect, the present invention provides a medical appliance useful for bone repair, regeneration, maintenance and augmentation. Exemplary embodiments generally include a carrier matrix, an osteoinductive agent, and an osteoinductive enhancer for modulating the activity of the osteoinductive agent, wherein said osteoinductive agent and said osteoinductive enhancer are both integrated within the carrier matrix. In certain preferred embodiments, the osteoinductive agent is a growth factor such as TGF-β or a BMP, and the osteoinductive enhancer is a phytoestrogen, mycoestrogen, such as naringin. The carrier matrix is generally a biocompatible material. In some embodiments, it is a demineralized bone matrix.

In a second aspect, the present invention provides a composition useful for bone repair, regeneration, maintenance and augmentation. Exemplary embodiments generally include an osteoinductive agent, an osteoinductive enhancer capable of enhancing the in vivo activity of the osteoinductive agent, and a physiologically acceptable carrier.

In a third aspect, the present invention provides a bone repair, regeneration, maintenance, and augmentation kit for use in bone related surgical procedures. Exemplary embodiments generally include a bone matrix or a biocompatible matrix containing an effective amount of an osteoinductive agent, and an osteoinductive enhancer.

In a forth aspect, the present invention provides a method for repairing, regenerating, maintaining, and augmenting a bone site in a patient. Exemplary embodiments generally include the steps of applying an exogenous osteoinductive agent and an osteoinductive enhancer to a treatment site of a patient, wherein the enhancer is capable of enhancing the in vivo activity of the osteoinductive agent.

In a fifth aspect, the present invention provides a bone matrix formulation for use in bone repair, regeneration, maintenance, and augmentation. Exemplary embodiments generally include a demineralized bone matrix that has one or more osteoinductive agent(s) embeded in it, and an effective amount of an osteoinductive enhancer.

Other aspects and advantages of the invention will be apparent from the following description and the appended claims.

### BRIEF DESCRIPTION OF THE DRAWINGS

FIG. 1 shows the variability of DBM activity depending on formulation and batch. DBM activity test: Osteoinductivity of BMP-2 and DBM can be quantitatively analysis by using cell culture method. Pre-myoblast cell line C2C12 was used for test BMP-2 induced ALP activity. Activity of DBM from different tissue banks or from same bank but different batches very significantly. Osteoinductive Index (OI) of 20 random selected DBM from tissue bank was listed in Table 1

FIG. 2 shows a dose response of alkaline phosphate (ALP) induction of active DBM in vitro. The assay was standardized by mixing varying amounts of inactive DBM into five lots of active DBM from the same bone bank. A proportional osteoinductive response was observed.

FIG. 2 shows a structure of naringin.

FIG. 3 shows a dose-dependency comparison of BMP-2 and the osteoinductivity enhancing effect of naringin. C2C12 was plated in 96-well culture plate with density of 12.5K/well in 10% FBS/DMEM for 5 hours attachment. Medium was changed into 1% testing medium followed by adding different amount of BMP-2 and/or naringin solution. Cells were incubated at 37° C. for another 48 hours. Cell membrane associated ALP activity was tested by standard ALP assay. BMP2 dose dependently increased ALP activity. Naringin itself had no effect on ALP activity, When naringin added to BMP-2, naringin dose dependently increase BMP-2 induced ALP activity.

FIG. 4 shows a biphasic behavior of naringin. C2C12 was plated in 96-well culture plate with density of 12.5K/well in 10% FBS/DMEM for 5 hours attachment. Medium was changed into 1% testing medium. Forty nano-gram of rhBMP-2 in 10 μl was added in every well and 1-1600 nM of naringin was added 10 minutes later. Naringin concentration at 800 nM exhibited maximal enhancing ALP effect. The sequence and time interval between the addition of growth factor and enhancer is also critical to their biological effect.

FIG. 5 shows that estrogen receptor antagonist ICI partially block naringin enhancing BMP-2 effect.

FIG. 6 shows cell proliferation by naringin. MTT was used for cell proliferation assay. MTT [3-(4,5-dimethylthiazol-2-yl)-2,5-diphenyltetrazolium bromide] assay is based on the ability of a mitochondrial dehydrogenase enzyme from viable cells to cleave the tetrazolium rings of the pale yellow MTT and form a dark blue formazan crystals which is largely impermeable to cell membranes, thus resulting in its accumulation within healthy cells. Rat bone marrow derived stem cells were selected by pro-plating methods. Cells used in this study were from passages 3-4. 1×10⁷ viable cells/mL in culture media containing 10% FCS were dispensed 100 μL per well in 96 well flat bottomed tissue culture plates. Cells were allow to grow with and without naringin and incubated plates in a humidified CO₂ incubator at 37° C. for 48 h. 10 μL of MTT solution were added to all wells of 48 h cultured stem cells and incubate for 4 h at 37° C. After washing cells 3 times with PBS, dark blue dye in cells were dissolved with DMSO. After a few minutes at room temperature read plates using a plate reader in dual wavelength measuring system, at 540 nm and a reference wavelength of 630 nm.

FIG. 7 shows an exemplary protocol for covalently bonding naringin to a collagen matrix.

FIG. 8 shows the activity profile for a collagen sponge matrix impregnated with or without naringin and BMP-2. Bovine tendon derived type I collagen sponge was used as BMP-2 carrier for in vivo osteoinductivity assays. Collagen sponge was either blended with naringin solution or covalently crosslinked naringin to collagen sponges. Different doses of BMP-2 were added to the matrices before implantation. Samples were placed intramuscular in thigh muscle. Ten Fisher 344 rats with 180 g in weight were used in this study. Implants were be retrieved 28 days postoperatively. Explants were dissected free of muscle flaps and cut into halves. One half of each explant was used for alkaline phosphatase assay.

FIG. 9 left panel shows the histology of bone formation intramuscularly. Right panel shows a sample fixed in 10% neutral buffered formalin solution for 24 hours and decalcified with a decalcifying solution (Stephens Scientific, Riverdale, N.J.) for 48 hours. The decalcified explant will be paraffin-embedded and sectioned with a 5 mm microtome. The sections will be later stained with Safarin-O and H&E for cartilage and bone and examined under light microscopy.

FIG. 10 shows the result of bone graft using DBM with and without addition of naringin.

### DETAILED DESCRIPTION

### 1. Basis for the Present Invention

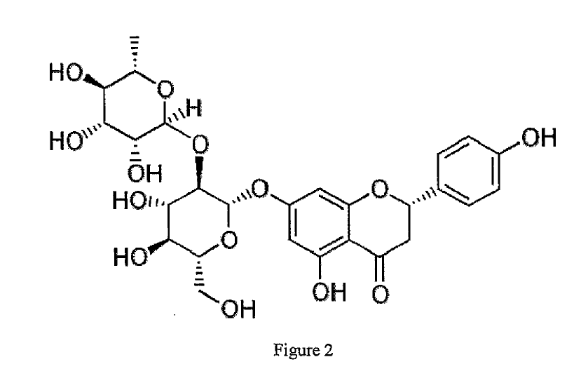

One of the inventors had previously demonstrated that a particular bioflavonoid, naturally originating from citrus fruits, naringin (FIG. 2), is able to enhance collagen synthesis in animals which are exposed to catabolic agents, such as the corticosteroids (Bavetta and Nimni, Am J Physiol 206: 179-182, 1964, the content of which is incorporated herein by reference). Under these circumstances, dietary naringin is able to increase collagen synthesis around subcutaneous implants.

More recently the inventors have discovered that bioflavonoids obtained from grape seeds (polyanthocyanidins) are also able to enhance collagen synthesis by cultured fibroblasts and in the skin after topical application (Han and Ninmi, Connect Tissue Res. 2005; 46(4-5):251-7, the content of which is incorporated herein by reference). Last year Wong and Rabie (Wong and Rabie, Biomaterials 27, 2006, 1824-1831 and J Orthop Res., 2006, November; 24(11):2045-50, both are incorporated herein by reference) were able to demonstrate both in vitro and in vivo that naringin could significantly enhance biosynthetic abilities of bone cells as well as the deposition of new bones at sites of bone repair in a cranial defect model in rats. In their animal model experiments, they achieved their results by implanting a collagen sponge soaked in a 10% solution of naringin at a site of cranial repair. Wong and Rabie concluded that naringin delivered by a collagen matrix carrier has osteoinductive capability in increasing new bone formation locally and suggests that collagen matrix laced with naringin is a promising bone graft material formulation. The data suggested that naringin might be a plant derived osteogenic agent.

Surprisingly, the inventors discovered that collagen sponges soaked in naringin, when implanted subcutaneously in rats, do not induce new bone, whereas collagen sponges which, in addition to naringin, contained growth factors (BMP2 and/or BMP7) did induce bone in the inventors' subcutaneous or intramuscular model.

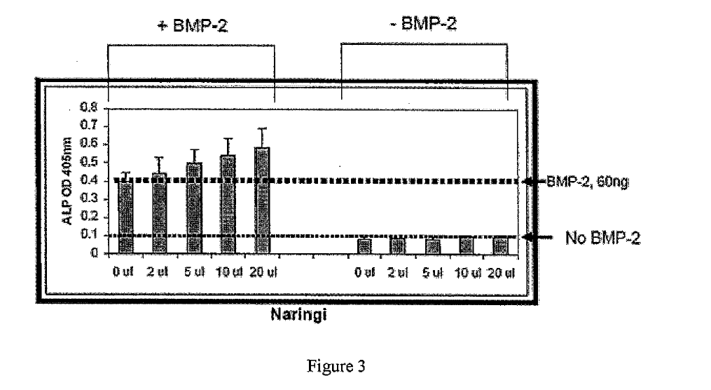

In addition, specimens of DBM (human demineralized bone discarded for use because of their low biological activity), when mixed with naringin, induced the formation of significant amounts of new bone subcutaneously. Because of their low content of growth factors, such DBM specimens did not by themselves induce subcutaneous bone. Evaluation using the standard protocol for investigating osteoinductivity, which relies on the ability of animals (usually rats) to form bone subcutaneously or intramuscularly following implantation of a test material, clearly indicates that naringin alone with a collagen carrier is not sufficient to induce bone. Based on these surprising discoveries, the inventors concluded that specific growth factors are needed in addition to naringin for bone to form (FIG. 3).

Thus, in essence, the present invention is based on the unexpected discovery that bioflavonoids such as naringin has the ability to enhance the osteoinductivity of osteoinductive agents such as growth factors and bone morphology proteins. More specifically, the inventors have advanced the art by discovering that naringin is not an osteogenic agent, as previously believed in the art, but is more appropriately characterized as an osteoinductive enhancer, i.e. it enhances the osteoinductivity of osteoinductive agents such as bone morphology proteins and certain growth factors. By adding naringin to sites where there is endogenous growth factors, bone growth rate is potentiated or enhanced.

It will be recognized by one skilled in the art that osteoinductive enhancers are particularly useful in situations where fracture healing is compromised, such as in older people where biosynthetic abilities may be declining, or in situations where not sufficient bone is available for repair. It will also be understood by one of ordinary skill in the art that the addition of growth factors (natural or synthetic) to the naringin containing preparation is essential for osteoinduction. The inventors' bioassay conducted in rats found that no bone was formed when only naringin was added to a collagen carrier.

In accordance with the discovery of the present invention, the inventors have devised methods, medical appliances, and bone matrix formulations utilizing the discovery. For example, matrices of biocompatible materials, such as collagen matrix, when combined with (adsorbed or covalently bound) naringin and suitable growth factors (preferably of the bone morphogenetic family, or BMP's) can become suitable devices to for enhancing bone formation in humans or animals.

In another example, naringin, a citrus bioflavonoid, can also be added to demineralized bone matrix of low or marginal biological activity, to greatly enhance its bone forming potential. Prior art literature has heretofore suggested that similarly to statins, agents commonly used to lower cholesterol, bioflavonoids may inhibit an enzyme (hydroxymethylglutaryl coenzyme A reductase) a rate limiting enzyme in the mevalonate pathway (Mundy et al., 1999 Dec. 3; 286(5446):1946-9). Naringin, a bioflavonoid which has an antioxidant as well as cholesterol lowering effect has a similar reductase inhibitor effect raising the possibility that it may also activate a BMP-2 promoter and, thereby, increase growth factor biosynthesis.

While not intending to be bound by any particular theory, the surprise finding of the present invention that naringin showed no effect in the intramuscular bone inducing assay, coupled with the fact that it works synergistically with exogenous BMP's suggests that it is more likely that bioflavonoids, in particular naringin, are effective due to stabilizing, through a direct chemical interaction, or with supplemental growth factors, at any of the levels in which they are encountered in tissues after administration (extracellularly, bound to receptors or membranes, intracellularly, etc).

Since DBM is used clinically in a large number of applications (current market estimated to be around $200 million armually) and since in many cases the biological activity of some of the preparations is low, and sometimes even questionable, it is an advantage of the present invention that addition of naringin and other osteoinductive enhancers could greatly enhance the osteogenic potential of these products. The same can be said of preparations which rely on the use of recombinant or naturally obtained growth factors, which are becoming increasingly used during the course of surgical procedures.

In addition, novel matrices, containing a host of growth factors, similar to the ones described can be further developed using the approach outlined. After the growth factors are stabilized with naringin or other bioflavonoids, their biological activity can be greatly enhanced, and the dose required in such applications significantly reduced, thereby, overcoming the linear proportion osteoinductivity problem of prior art matrices.

It will be readily understood by those skilled in the art from the foregoing discussion that application of naringin alone, without growth factors, cannot provide a reliable device for enhancing bone repair. Numerous industrial and medical applications may flow from this discovery. As discussed above, novel composite preparations such as those described in the present invention can be of significant benefit.

In a series of studies, it has been observed that adult rats show a marked decline in their ability to respond to growth factors with increasing age. This further suggests a need for local supplementation of growth factor to counter the aging effect, which highlights the need for the medical application embodiments of the present invention.

Now, a detailed description of the various aspects and exemplary embodiments will be discussed.

### 2. Embodiments of the Present Invention

In a first aspect, the present invention provides a medical appliance useful for bone repair, regeneration, maintenance and augmentation. Embodiments in accordance with this aspect of the present invention generally include a carrier matrix; an osteoinductive agent; and an osteoinductive enhancer for modulating the activity of the osteoinductive agent.

As used herein, the phrase “medical appliance” refers to an object or an article of manufacture for use in any of a number of medical applications. In preferred embodiments, medical appliance of the present invention may function as inserts or implants for substituting body parts or for facilitating the repair, regeneration, maintenance, and augmentation of body parts. In cases where the intended application is for bone repair, regeneration, maintenance, and augmentation, medical appliance of the present invention is applicable across all types of bones. Examples of applications in which an appliance of the present invention may be used include bone fracture repair, spinal fusion, cranial maxillofacial surgery, bone and cartilage defects, or any other types of procedures that require formation of new bones, but are not limited thereto. It will be appreciated by one of ordinary skill in the art that other types of applications such as dental augmentation procedures are also within the scope the present invention.

To provide physical structure, appliances in accordance with the present invention has a carrier matrix. The matrix is preferably made of a biocompatible material. Other factors for choosing a material suitable for the matrix may include considerations for the characteristics such as porosity, density, malleability, price, rate of resorbtion, biodegradability, surface charge, wettability, and degradation products. More preferably, the material is one that is suitable for subcutaneous implantation.

Exemplary construction materials for the matrix may include fibrillar collagen, any demineralized bone matrix formulation known in the art, ceramics, hydroxyapatites, crosslinked collagen or gelatin, glycoaminoglycan crosslinked networks, collagen coated ceramics, PLA, PGA, mixed copolymers, or a combinations thereof, but not limited thereto. Other biocompatible materials known in art may also be advantageously employed.

Although it is preferred that the carrier matrix is inert with regard to the recipient, in certain embodiments, a bioactive matrix may also be used. When the carrier matrix is a low activity or inactivated DBM, the resulting appliance may exhibit certain advantageous characteristics. An embodiment which uses low activity DBM as the matrix may enjoy a low cost, and an amplifiable/activatable bioactivity to be modulated by an osteoinductive enhancer of the present invention.

Osteoinduction is the process by which osteogenesis is induced. It is a phenomenon regularly seen in any type of bone healing process. Osteoinduction implies the recruitment of immature cells and the stimulation of these cells to develop into preosteoblasts. In a bone healing situation such as a fracture, the majority of bone healing is dependent on osteoinduction.

Osteoconduction means that bone grows on a surface. This phenomenon is regularly seen in the case of bone implants. Implant materials of low biocompatibility such as copper, silver and bone cement shows little or no osteoconduction.

Osseointegration is the stable anchorage of an implant achieved by direct bone-to-implant contact. In craniofacial implantology, this mode of anchorage is the only one for which high success rates have been reported.

Therefore, the phrase “osteoinductive agent” as used herein refers to any molecule or chemical that is capable of effecting the process of osteoinduction.

To form an appliance of the present invention, any known osteoinductive agent may be suitably chosen, depending on the intended use, compatibility with the carrier matrix, and the cooperative interaction with the osteoinductive enhancer Exemplary osteoinductive agents include bone morphology proteins, and growth factors. Preferred bone morphology proteins include BMP-2, BMP-6, BMP-7, BMP-9, BMP-12, and BMP-13, but are not limited thereto. Preferred growth factors are one that is selected from the transforming growth factor family, such as TGF-β, but are also not limited thereto.

The growth factors and morphology proteins described above may be obtained from any number of sources by any techniques known in the art, including crude extracts, purified concentrates, and recombinantly produced, but are not limited thereto. It will be appreciated by a person of ordinary skill in the art that these proteins and growth factors may have various isoforms which are also applicable. Common modifications and derivatives may also be included for convenience or for optimized performance. Thus, when referring to an osteoinductive agent, its various isoforms and common derivatives are also contemplated.

In certain embodiments, there may be more than one active osteoinductive agent included in the appliance. This may be intentional or non-intentional. In either case, it does not alter the spirit of the present invention and is considered to be encompassed within the scope of the present invention.

As set forth in the background, protein-based osteoinductive agents are prone to degradation and lose their activity over time. It is an unexpected discovery of the present invention that bioflavonoids such as naringin, may function not as oesteoinductive agents, but as osteoinductive enhancers.

As used herein, the phrase “osteoinductive enhancer” refers to any compound or entity that, when combined together with an osteoinductive agent, may act to enhance or prolong the activity of the osteoinductive agent.

In certain preferred embodiments, the osteoinductive enhancer may be selected from a phytoestrogen, a mycoestrogen, a derivative thereof, or an analogue thereof.

As used herein, “phytoestrogen” refers to a diverse group of naturally occurring non steroidal plant compounds that because of their structural similarity with estradiol (17β-estradiol), have the ability to cause estrogenic or/and antiestrogenic effects. Flavonoids such as naringin has been shown to have estrogen-like activity, hence, is a member of this class (Effenberger et al., Journal of Steroid Biochemistry & Molecular Biology 96, 2005, 387-399, the content of which is incorporated herein by reference). Mycoestrogens are structurally and chemically similar to phytoestrogens, except that they are derived from fungi.

In some embodiments, the osteoinductive enhancer may be selected from a bioflavonoid. More preferably, it may be selected from a flavone, an isoffavone, a flavonone, a chalcone, or a polymer thereof. It may also be selected from naringin, naringenin, a derivative thereof, or a combination thereof.

Phytoestrogens include lignan, isoflavone, flavone, and coumestan compounds, and their metabolites, such as equol. The lignans, isoflavones, flavones, and coumestans have structures that are conformationally similar to the structure of 17-p-estradiol, thus they act-as estrogen analogues with respect to estrogen receptor binding sites. (C. L. Hughes et al., Progress in the Management of the Menopause, B. G. Wren [ed.], The Parthenon Publishing Group, pp. 30-39 1997, the content of which is incorporated herein by reference). Some specific examples are daidzin, genistin, and glycitin

Phytoestrogens are plant-derived substances whose structure results in a chemical nature similar to endogenous estrogens of humans and other members of the animal kingdom. Phytoestrogens are categorized into four main groups and these are further subdivided. The most chemically efficacious and structurally similar to estrogen, are the isoflavones. With the structural similarity allows the isoflavones to act upon the estrogen receptors within the body.

Recently, Kousteni et al (Kousteni S, et al., Mol Cell Biol. 2007 February; 27(4):1516-30.). first revealed the existence of a large signalosome in which inputs from the estrogen receptor, kinases, bone morphogenetic proteins, and Wnt signaling converge to induce differentiation of osteoblast precursors. Estrogen receptor can either induce it or repress it, depending on whether the activating ligand (and presumably the resulting conformation of the receptor protein) precludes or accommodates ERE-mediated transcription. Thus, it is expected that phytoestrogens and mycoestrogens will have the same oesteoinductive enhancing property of naringin to a varying degree.

In certain preferred embodiments, the combination of an osteoinductive agent and an osteoinductive enhancer results in a synergistic effect, i.e. a higher level of biological activity than either one can achieve independently.

Referring to FIG. 3, in the absence of the osteoinductive enhancer, naringin, the osteoinductive agent, BMP-2, only shows a 0.1 activity in the alkaline phosphate (ALP) (see Han, et al., J Orthop Res. 2003 July; 21(4):648-54 for details of the assay, the content of which is incorporated here by reference) assay. When naringin is added, however, BMP-2 activity is increased four-fold to 0.4. On the other hand, naringin by itself has no osteoinductivity at all. Therefore, the combination of naringin and BMP-2 is considered to show “synergistic effect”.

In forming a medical appliance in accordance with embodiments of the present invention, it is preferred that the osteoinductive agent and the osteoinductive enhancer are both integrated within the carrier matrix to form an appliance of the present invention. In the context of the present invention, integration simply means that the two components are spatially located within the confine of the carrier matrix. No particular physical or chemical interaction is required for “integration”. However, without being bound to any particular theory, it is hypothesized that at least part of the osteoinductive enhancing effect of the enhancer derives from being able to delay or prevent degradation of the osteoinductive agents, i.e. stablizing the biological activity of the agents. Because molecular binding is known as a common direct mechanism for stablizing an otherwise labile biomolecule, in certain embodiments, it is preferred that the osteoinductive agent and the osteoinductive enhancer are allowed to be mixed within the matrix.

In a preferred embodiment, the osteoinductive agent and the osteoinductive enhancer are mixed in a ratio of from about 0.01:1.0 to about 100:1.0.

In other embodiments, the osteoinductive agent is entrapped within or on the surface of the carrier matrix via adsorption, covalent cross-linking, hydrophobic interaction, ionic interaction, hydrophilic interaction, or a combination thereof.

In a second aspect, the present invention provides a composition useful for bone repair, regeneration, maintenance, or augmentation. Embodiments according to this aspect of the present invention generally include an osteoinductive agent, an osteoinductive enhancer capable of enhancing the in vivo activity of the osteoinductive growth factor; and a physiologically acceptable carrier.

The osteoinductive agent and the osteoinductive enhancers are same as described above.

A physiologically acceptable carrier is generally one that does not elicit an adverse reaction in the recipient. Common choices of material for forming the carrier are same as described above for the carrier matrix. In a preferred embodiment, a composition of the present invention will allow delivery of the osteoinductive agent and enhancer to allow reach to an ectopic location.

In certain embodiments, the physiological carrier is capable of extended release and stably storing the osteoinductive agents and enhancers.

In a third aspect, the present invention provides a bone repair, regeneration, maintenance, and augmentation kit for use in bone related surgical procedures.

Embodiments according to this aspect of the present invention generally include a bone matrix or a biocompatible matrix containing an effective amount of an osteoinductive agent; and an osteoinductive enhancer.

This aspect of the present invention pays particular attention to commercial and practical concerns. One major application of bone matrices is to provide for bone grafting materials. In this context, bone matrices may be provided in a number of configurations, including in power form, in premixed putty, or any other convenient form of packaging the ingredients.

In general, embodiments according to this aspect of the present invention have substantially the same osteoinductive agents and enhancers as described above. In certain preferred embodiment, the bone matrix is one selected from a DBM commonly available in the art. The addition of the enhancer is capable of substantially upgrading the bioactivity of an otherwise less valuable product. Thus the value added potential of kits of the present invention is significant.

In some embodiments, the enhancer is provided with the matrix as an integral product. Such configuration has the advantage of being easy to package and offer convenience for the user.

In some other embodiments, the enhancer may be provided as a solubilized product in a stablizing liquid medium. Alternatively, it may be lyophilized and provided in powder form to be rehydrated prior to use.

In a preferred embodiment, the bone matrix is a low activity DBM. Such DBM are usually discard as non-active, and, therefore, of low commercial value. However, the addition of an enhancer in accordance with embodiments of the present invention rescues an otherwise discarded product. Embodiments according to this aspect of the present invention has the advantage that different osteoinductive agents and inducers can be produced independently and then recombined in different combinations to meet the different needs of end users while still enjoying the benefit of scale-of-economy on the manufacturing side.

In a fourth aspect, the present invention provides a method for repairing, regenerating, maintaining, and augmenting a bone site in a patient. Embodiments according to this aspect of the present invention generally include the steps of applying an exogenous osteoninductive agent and an osteoinductive enhancer to a treatment site of a patient. In this aspect, the combination of osteoinductive agent and enhancer should be chosen such that the osteoinductive agent is compatible with the recipient and the enhancer is synergistic or at least complimentary with the agent.

Choices for the osteoinductive agents and enhancers are substantially same as described above. It will be appreciated by one of ordinary skill in the art that by virtual of the unexpected discovery of the present invention, the combination of exogenous osteoinductive agent and enhancer, when applied to a site that does not have endogenous osteoinductive agents, may result in ectopical bone growth at such sites. Thus, embodiments of this aspect of the present invention offers the advantage that a great degree of control may be achieved in the delivery of the desired treatment effect, particularly in situations where bone growth is desired around a transplanted organ, tissue, or bodily structure. Other types of treatment procedures, including augmentation, repair, regeneration and maintenance are all benefited.

In a fifth aspect, the present invention provides a bone matrix formulation for use in bone repair, regeneration, maintenance, and augmentation.

Embodiments according to this aspect of the present invention generally include a demineralized bone matrix having embedded therein one or more osteoinductive agents; and an effective amount of an osteoinductive enhancer.

It will be recognized by one of ordinary skill in the art that this aspect of the present invention is substantially a variation of the other embodiments of the present invention. However, the unexpected discovery of the present invention has opened up the possibility of novel formulations utilizing various different combinations of osteoinductive agents and enhancers to meet different application needs.

The various exemplary embodiments as well as other embodiments not specifically described herein will have many advantages. For example, in those embodiments where an enhancer is used together with a low activity bone matrix, the formulation will achieve biological activities previously only achievable with much higher quality demineralized bone matrices. Because the combination of exogenous osteoinductive agent and enhancer will have the ability to induce ectopical bone growth, procedures such as spinal bone fusion may be greatly facilitated. Grafted bone material may also be encouraged to achieve osteoconduction and integration.

Having generally described this invention, a further understanding can be obtained by reference to certain specific examples which are provided herein for purposes of illustration only and are not intended to be limiting unless otherwise specified.

### EXAMPLES

All references cited in the following examples are each incorporated by reference.

### Example 1

### Phytoestrogens Used for Treatment and Prevention of Osteoporosis

Estrogen and phytoestrogen have been reported to prevent bone loss in both postmenopausal women and ovariectomized (ovx) rats.

The hypothesis that flavonols might also be bioactive molecules, which may be able to counteract the deleterious effects of estrogen deficiency occurring during menopause, has been recently addressed by Horcajada-Molteni et al (Horcajada-Molteni, M. N., et al., J Bone Miner Res, 2000. 15(11): p. 2251-8), who demonstrated that rutin, a glycoside derivative of quercetin, one of the major flavonols, inhibits ovariectomy-induced osteopenia in female rats. Loss of estrogens or androgens accelerates the effects of aging on bone by decreasing defense against oxidative stress and such process can be reversed by estrogens or androgens in vivo as well as in vitro (Syed, F. A., et al., Osteoporos Int, 2008.; Juttner, K. V. and M. J. Perry, Bone, 2007. 41(1): p. 25-32; Waarsing, J. H., et al., J Orthop Res, 2006. 24(5): p. 926-35).

Estrogens exert their physiological effects on target tissues by interacting with estrogen receptors (ERs), which are members of the superfamily of ligand regulated nuclear transcription factors (Monroe, D. G., et al., J Musculoskelet Neuronal Interact, 2003. 3(4): p. 357-62; discussion 381.). Two ERs have been discovered to date, ER-α and ER-β. Both receptors have been identified in osteoblasts and osteoclasts as well as in their precursors (Parikka, V., et al., Eur J Endocrinol, 2005. 152(2): p. 301-14.), but the precise roles of ER-α and ER-β in bone turnover remains to be fully elucidated. An et al. (An, J., et al., J Biol Chem, 2001. 276(21): p. 17808-14) have found that estrogens and phytoestrogens are more effective at transcriptional repression in the presence of ER-α compared with ER-β. Richard (Rickard, D. J., et al, J Cell Biochem, 2003. 89(3): p. 633-46) reported genistein behaves as a weak E(2) agonist in osteoblasts and can utilize both ER-α and ER-β.

Systemically administered 17-estradiol (E2) has been found to enhance bone formation in animals. Although the precise mechanism of E2-induced bone formation is not clear (Raisz, L. G., Ciba Found Symp, 1988. 136: p. 226-38.), the BMP-2 gene is a potential target for estrogens. In fact, E2 has been shown to upregulate BMP-2 mRNA expression in the murine osteogenic cell line MN7. In addition, E2 up-regulates mouse BMP-2 gene expression in mouse bone marrow MSCs, which express both ER-α and ER-β. Moreover, ovariectomy decreased basal levels of BMP-2 mRNA in the mouse MSCs. Finally, when systemically treating mice suffering from osteoporosis after ovariectomy with BMP-2, bone mass was restored to its normal values and MSCs restored their proliferation and differentiation activity. These findings indicate that estrogens may promote bone formation by stimulating BMP-2 gene transcription. Estrogens regulate BMP-2 gene transcription in MSCs and C3H10T1/2 cells. E2 activates BMP-2 gene transcription by recruiting ER-α. and ER-β to a variant estrogen responsive element (ERE) binding site in the BMP-2 promoter. These findings suggest in addition to its well-recognized inhibitory effect on bone resorption, estrogens may also promote bone formation by enhancing production of BMP-2 (Kousteni, S., et al., Mol Cell Biol, 2007. 27(4): p. 1516-30.).

Flavonoids including naringin and other phytoestrogens belong to a family of plant derived polyphenols. The primary focus has been placed on the antioxidant properties of these flavonoids, there is an emerging view that flavonoids, as well as their in vivo metabolites, do not function as conventional hydrogen-donating antioxidants, but may instead exert modulatory actions in cells via their actions at the protein kinase and lipid kinase signaling pathways. Flavonoids, and more recently their metabolites, have been reported to function at the phosphoinositide 3-kinase (PI 3-kinase), Akt/protein kinase B (Akt/PKB), tyrosine kinases, protein kinase C(PKC), and mitogen activated protein kinase (MAP kinase) signaling cascades. Inhibitory or stimulatory effects at these pathways are likely to modulate cellular functions profoundly, via alterations of the phosphorylation states of target molecules, and via the modulation of gene expression.

Dietary glycosides are converted to aglycones (such as quercetin) in the large intestine, in reactions catalyzed by the glycosidases generated by intestinal bacteria (Ross, J. A. and C. M. Kasum, Annu Rev Nutr, 2002. 22: p. 19-34.). Prouillet et al. (Prouillet, C., et al, Biochem Pharmacol, 2004. 67(7): p. 1307-13) reported that quercetin and kaempferol induced an increase in alkaline phosphatase activity in MG-63 human osteoblasts via the activation of the estrogen receptor, and Miyake et al (Kanno, S., S. Hirano, and F. Kayama, Toxicology, 2004. 196(1-2): p. 137-45.) reported on the promoting effect of kaempferol on the differentiation and mineralization of a murine pre-osteoblatic cell line. Combination of genistein and zinc can synergistically enhance gene expression and mineralization in osteoblastic cells (Uchiyama, S, and M. Yamaguchi, Int J Mol Med, 2007. 19(2): p. 213-20) Daidzein may be able to enhance the bone differentiation and mineralization and prompt the bone formation in the early growing stage and the late growing stage of osteoblasts (Ge, Y., et at, Yakugaku Zasshi, 2006. 126(8): p. 651-6). Daidzin, genistin, and glycitin may modulate differentiation of MSC to cause a lineage shift toward the osteoblast and away from the adipocytes, and could inhibit adipocytic transdifferentiation of osteoblasts (Li, X. H., et al, Acta Pharmacol Sin, 2005. 26(9): p. 1081-6.)

Geinistein can stimulate bone-nodule formation and increase the release of osteocalcin in rat osteoblasts. The effects, like those induced by 17 beta-estradiol, are mediated by the estrogen receptor dependent pathway. Daidzein also can stimulate bone-nodule formation and increase the release of osteocalcin in rat osteoblasts, but it is not, at least not merely, mediated by the estrogen receptor dependent pathway (Chang, H., et al, Biomed Environ Sci, 2003. 16(1): p. 83-9).

Daidzein, a natural isoflavonoid found in Leguminosae, has received increasing attention because of its possible role in the prevention of osteoporosis. Daidzein (2-50 microM) increased the viability (P<0.05) of osteoblasts by about 1.4-fold. In addition, daidzein (2-100 microM) increased the alkaline phosphatase activity and osteocalcin synthesis (P<0.05) of osteoblasts by about 1.4- and 2.0-fold, respectively. Alkaline phosphatase and osteocalcin are phenotypic markers for early-stage differentiated osteoblasts and terminally differentiated osteoblasts, respectively. These results indicated that daidzein stimulated osteoblast differentiation at various stages (from osteoprogenitors to terminally differentiated osteoblasts). Effect of daidzein on bone morphogenetic protein (BMP) production in osteoblasts was also investigated, the results indicated that BMP2 synthesis was elevated significantly in response to daidzein (the mRNA increased 5.0-fold, and the protein increased 7.0-fold), suggesting that some of the effects of daidzein on the cell may be mediated by the increased production of BMPs by the osteoblasts (Jia, T. L., et al., Biochem Pharmacol, 2003. 65(5): p. 709-15).

Recently, the idea of the existence of a large signalosome was proposed (Kousteni, S., et al, Mol Cell Biol, 2007. 27(4): p. 1516-30). Signalosome is a complex formed from estrogen receptor and various ligands. It was proposed to have the function to converge the inputs from the ER, kinases, bone morphogenetic proteins, and Wnt signaling to induce differentiation of osteoblast precursors. ER can either induce it or repress it, depending on whether the activating ligand (and presumably the resulting conformation of the receptor protein) precludes or accommodates ERE-mediated transcription. Naringin and other phytoestrogens may fall into this pathway to regulate the osteogenic precursor differentiation.

### Example 2

### Phytoestrogen Including Naringin as Bone Graft Device

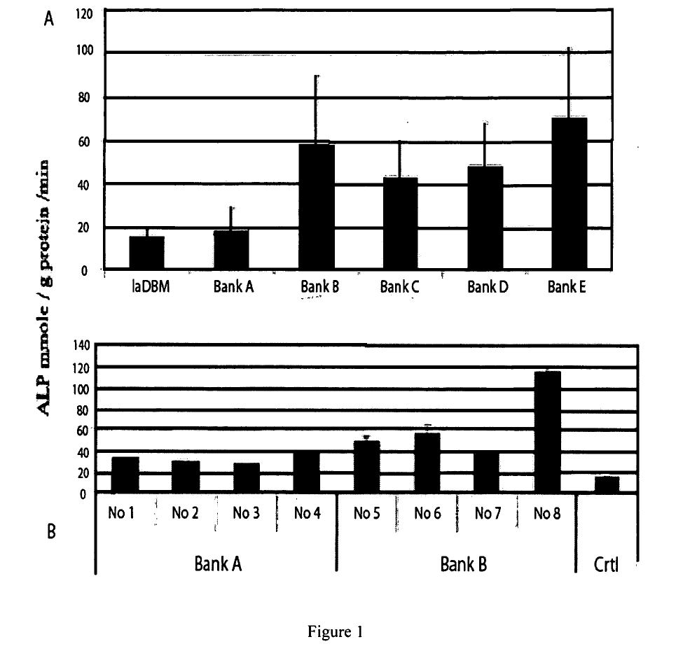

Naringin is a polyphenol present in citrus. By means of alkaline phosphatase activity, we have shown that naringin exhibits a significant induction of differentiation in osteoprogenitor cells (C2C12) (FIG. 1). Alkaline phosphatase is phenotypic markers for early-stage differentiated osteoblasts and terminally differentiated osteoblasts, respectively, our preliminary results indicate that naringin stimulate osteoblast differentiation.

### Example 3

### Additive Effect of Naringin on BMP-2 for Osteoprogenitor Cell Differentiation

Induction of differentiation by naringin is associated with increased bone morphogenetic protein-2 (BMP-2) production. Addition of naringin to undifferentiated C2C12 increases the upregulation of alkaline phosphatase activity by BMP-2. Naringin itself is not enough for osteogenic induction. (see FIG. 3).

### Example 4

### Naringin Effect is Time and Dose Dependent

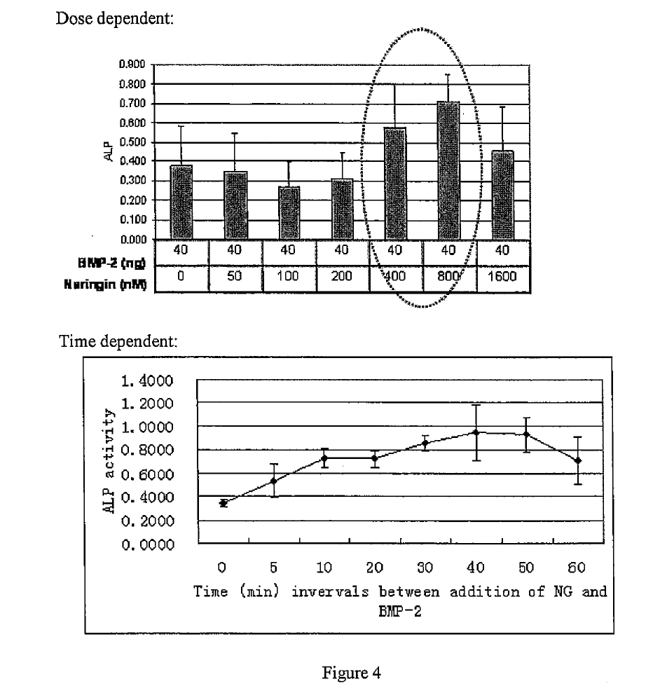

Naringin dose range in vitro on C2C12: around 12.5 to 2000 nM, lower than most phytoestrogen dose, but within the range of non-productive actions of estrogen derivatives. (see FIG. 4).

### Example 5

### Naringin Regulate Human BMP-2 in C1C12 is Partially Through Estrogen Receptor

Intracellular distribution of the estrogen receptor in skeletal myoblasts (C2C12) was determined by immuno-fluorescent staining (Milanesi, L., et al., J Cell Biochem, 2008). Naringin bind to estrogen receptor. Inhibition of estrogen receptor abrogate naringin effect. Naringin has been shown to stimulate osteogenesis both in vitro and in vivo. However, the mechanism by which naringin exerts its effects is still unclear. From our prelimianry study, there is evidence that naringin acts via estrogen-receptor (ER)-mediated signaling. Cells were cultured with naringin, in combination with 7α,17β-[9[(4,4,5,5,5-pentafluoropentyl)sulfinyl]nonyl] estra-1,3,5(10)-triene-3,17-diol (ICI182,780), a non-specific ERα and ERβ antagonist.

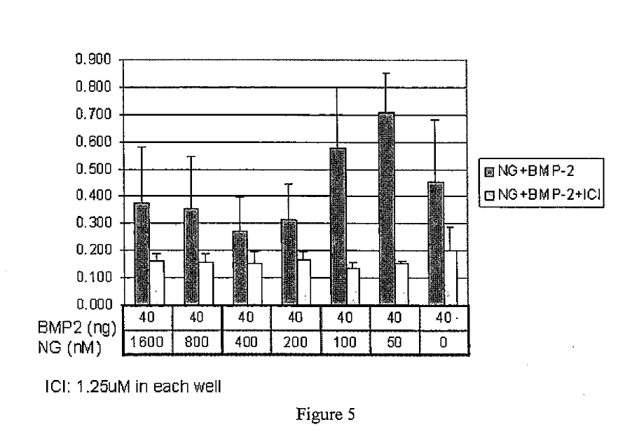

Referring to FIG. 5, results from these studies showed naringin enhances osteoblast differentiation partly through an ERalpha or ER-beta dependent pathway.

### Example 7

### Naringin is not a Mitogenic Factor for Stem Cells

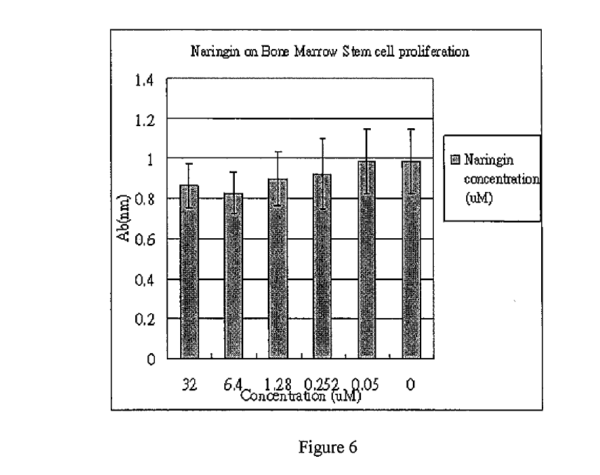

Dose dependent of naringin on bone marrow stem cell proliferation was studied. Cell number was assayed by MTT method. It was found that Naringin inhibits rat bone marrow cell proliferation. (see FIG. 6).

### Example 8

### Formulate Naringin Collagen Composite Materials

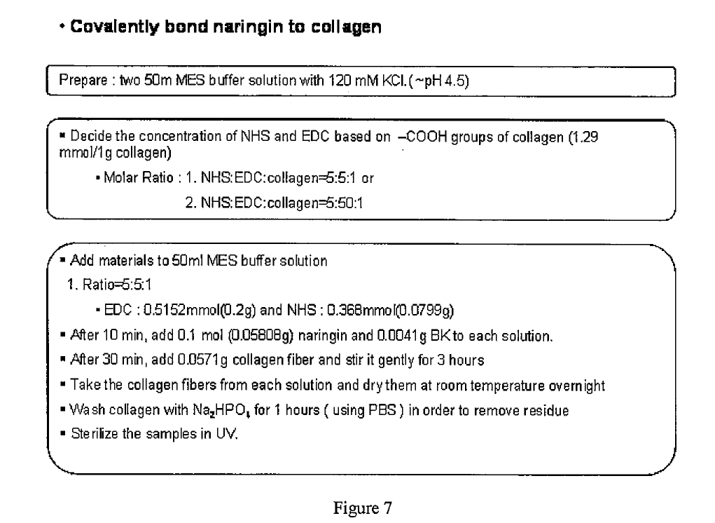

Two exemplary embodiments: (1) Naringin can be added to the carrier material by physical interaction (absorption); (2) Naringin can be controlled release from the scaffold by encapsulate, blended polymers or covalent bond to the material. (see FIG. 7)

### Example 8

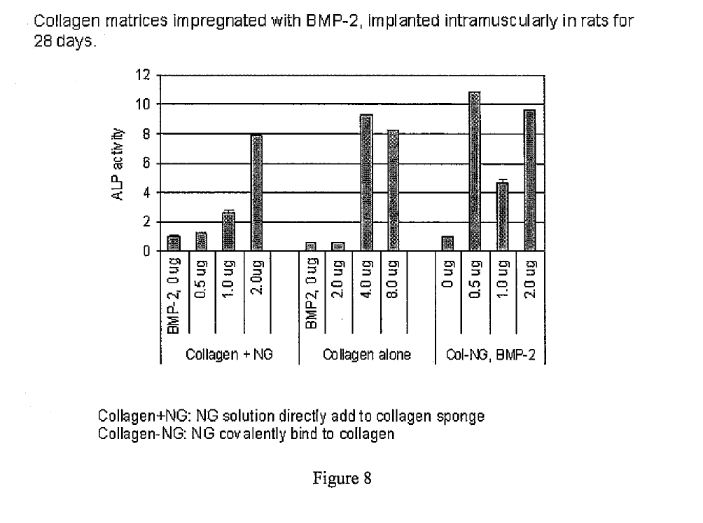

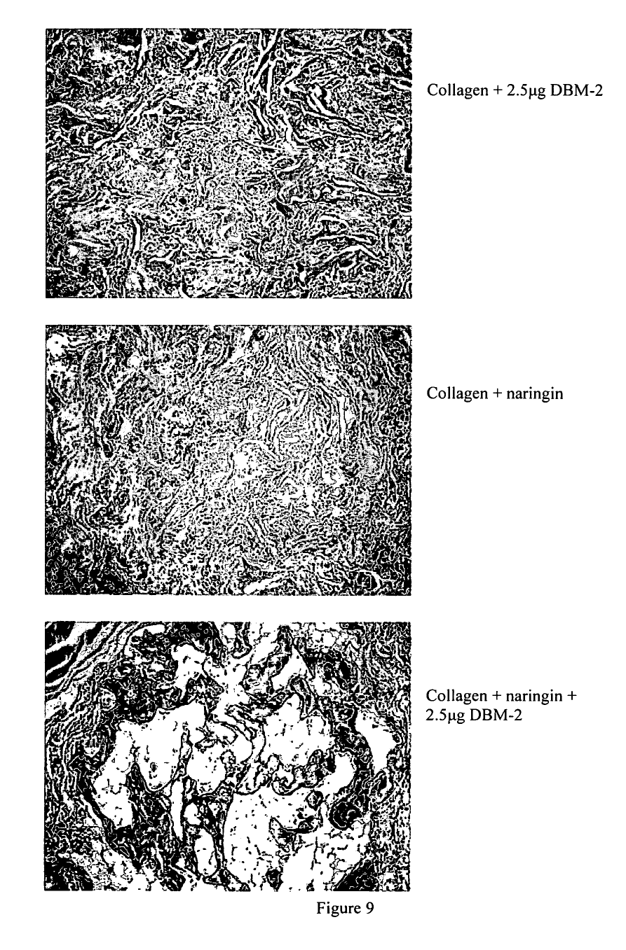

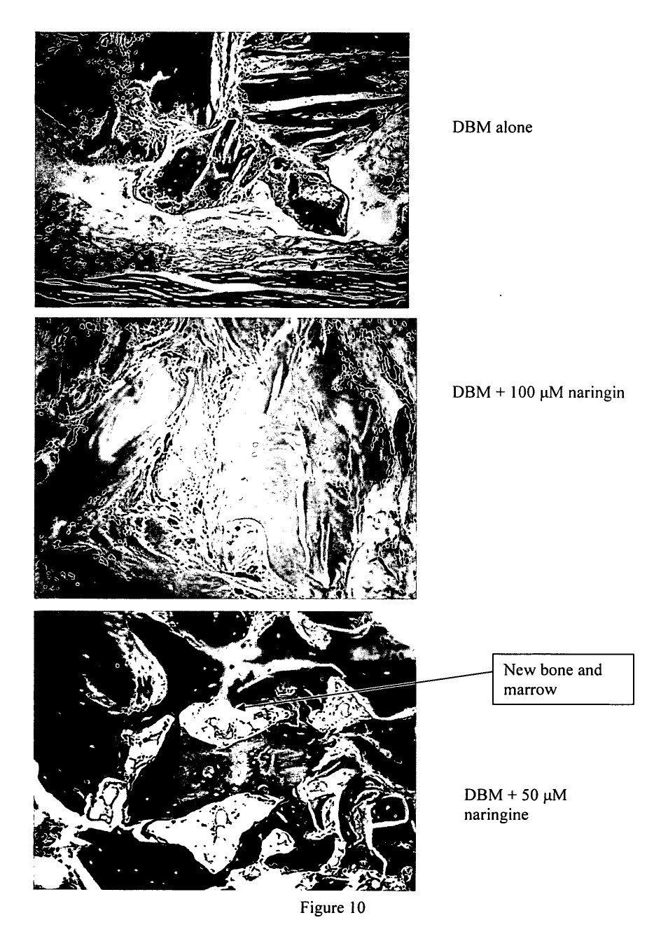

### Testing of Naringin Complex Materials as Bone Grafts In Vivo Bone Formation (Refer to FIGS. 8-10)

DBM Preparation:

Osteoinductive (OI) activity of human DBM obtained from Tissue Bank with particle size of 210-750 micrometers was tested in vitro with cell culture methods (Han, 2003). DBM were grouped according their OI score.

Two doses of naringin were used to treat DBM particles before implantation. In 50 mg of DBM, 50 micro liter of 100 micromolar or 50 micro of naringin/PBS solution was added into DBM before implantation.

### Surgery Procedures:

Muscle pouches were created in abdominal muscles bilaterally in nude rats (weighing 120-150 g each) at 6 sites by sharp and blunt dissection. Subsequently, fifty milligrams of three groups of DBM, two doses of naringin/PBS and with PBS alone, were packed into pouches and closed with a single absorbable suture. Implants were retrieved 28 days postoperatively. The recti abdomini muscles were excised as single muscle flaps and placed in cold PBS moisturized paper towels for radiography. Explants were dissected free of muscle flaps and cut into halves. One half of each explant were used for alkaline phosphatase assay and the other half were fixed in 10% neutral buffered formalin solution for 24 hours and decalcified with a decalcifying solution (Stephens Scientific, Riverdale, N.J.) for 48 hours. The decalcified explants were paraffin-embedded and sectioned with a 5 mm microtome. The sections were later stained with Safarin-O and H&E for cartilage and bone and examined under light microscopy.

### Results:

In vivo ALP activity and Bone Formation Score

| | in vivo OI | |
|---|---|---|
| DBM groups | score (1 to 4) | ALP units |
| DBM alone | 0.5 | 2.40 |
| DBM (100 uM NG) | 0.5 | 2.01 |
| DBM (50 uM NG) | 3.0 | 11.5 |

With optimized dose of naringin, DBM activity in regard of ectopic bone formation potential was significantly increased.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the appended claims

TABLE 1
Osteoinductivity Index of 20 Random DBM Samples

| DBM ID | OI Index |
|---|---|
| 1 | 0.02 |
| 2 | 0.18 |
| 3 | 1.62 |
| 4 | 0.92 |
| 5 | 0.13 |
| 6 | 0.37 |
| 7 | 0.53 |
| 8 | 0.50 |
| 9 | 2.58 |
| 10 | 0.26 |
| 11 | 0.07 |
| 12 | 0.05 |
| 13 | 0.45 |
| 14 | 0.16 |
| 15 | 0.24 |
| 16 | 0.05 |
| 17 | 0.22 |
| 18 | 0.16 |
| 19 | 0.04 |
| 20 | 0.54 |

OI: Osteoinductivity Index
OI = (ALP sample − ALP negative)/(ALP positive − ALP negative)

What is claimed is:

1. A medical appliance useful for bone repair, regeneration, maintenance, or augmentation, comprising:
a carrier matrix;
an osteoinductive agent; and
an osteoinductive enhancer for modulating the activity of the osteoinductive agent, wherein said osteoinductive agent and said osteoinductive enhancer are both integrated within the carrier matrix.

2. The appliance of claim 1, wherein said carrier matrix is a biocompatible material suitable for subcutaneous implantation.

3. The appliance of claim 2, wherein said carrier matrix is one selected from fibrilar collagen; low activity or inactivated DBM (Demineralized bone matrix) ceramics; hydroxyapatites; crosslinked collagen or gelatin; glycosaminoglycan crosslinked networks; collagen coated ceramics, PLA, PGA, missed copolymerscancellous scaffolds (mineralized or demineralized); particulate, demineralized, guanidine extracted, species-specific (allogenic) bone; specially treated particulate, protein extracted, demineralized, xenogenic bone; synthetic hydroxyapatites; polymers; hydrogels; starches; tricalcium phosphate, sintered hydroxyapatite, settable hydroxyapatite; polylactic acid; tyrosine polycarbonate; calcium sulfate; collagen sheets; settable calcium phosphate; polymeric cements; settable poly vinyl alcohols; polyurethanes; or a combination thereof.

4. The appliance of claim 2, wherein said osteoinductive agent is one selected from the TGF-β family, combinations thereof, or derivatives thereof.

5. The appliance of claim 2, wherein said osteoinductive agent is one selected from BMP-2, BMP-7, or a derivative thereof, or a combination thereof.

6. The appliance of claim 2, wherein said osteoinductive agent is a purified recombinant protein.

7. The appliance of claim 2 wherein said osteoinductive agent is a mixture of natural purified growth factors extracted in their active form from DBM.

8. The appliance of claim 2, wherein said osteoinductive enhancer is a bioflavonoid, a phytoestrogen, a mycoestrogen, a derivative thereof, or a combination thereof.

9. The appliance of claim 8, wherein said bioflavonoid is a flavone, an isoflavone, a flavonone, a chalcone, or a polymer thereof.

10. The appliance of claim 8, wherein said bioflavonoid is naringin, naringenin, a derivative thereof, or a combination thereof.

11. The appliance of claim 8, wherein said phytoestrogen is Daidzin, genistin, glycitin, or a combination thereof.

12. The appliance of claim 1, wherein said osteoinductive agent is entrapped within or on the surface of the carrier matrix via adsorption, covalent cross-linking, hydrophobic interaction, ionic interaction, hydrophilic interaction, or a combination thereof.

13. The appliance of claim 1, wherein the appliance is useful as an insert for treating bone fracture, spinal fusion, bone and cartilage defects, soft tissue augmentation, dental augmentation, or a combination thereof.

14. The appliance of claim 1, wherein said osteoinductive agent and said osteoinductive enhancer are present in the matrix in a ratio of from about 0.01:1.0 to about 100:1.0.

15. A composition useful for bone repair, regeneration, maintenance, or augmentation, comprising:
an osteoinductive agent;
an osteoinductive enhancer capable of enhancing the in vivo activity of the osteoinductive agent; and
a physiologically acceptable carrier.

16. The composition of claim 15, wherein said osteoinductive agent is a growth factor selected from the TGF-β family, combinations thereof, or derivatives thereof.

17. The composition of claim 15, wherein said osteoinductive agent is a Bone Morphology Protein selected from BMP-2, BMP-6, BMP-7, BMP-9, BMP-12, BMP-13, a derivative thereof, or a combination thereof.

18. The composition of claim 15, wherein said osteoinductive agent is a purified recombinant growth factor or bone morphology protein.

19. The composition of claim 15, wherein said osteoinductive enhancer is a bioflavonoid, a phytoestrogen, a derivative thereof, or a combination thereof.

20. The composition of claim 15, wherein said osteoinductive enhancer is a flavone, an isoflavone, a flavonone, a chalcone, or a polymer thereof.

21. The composition of claim 15 wherein said osteoinductive enhancer is phytoestrogen, mycoestrogen, a derivative thereof, or a combination thereof.

22. The composition of claim 15, wherein said osteoinductive enhancer is naringin, naringenin, daidzin, genistin, glycitin, a derivative thereof, or a combination thereof.

23. The composition of claim 15, wherein said carrier is a low activity deminiralized bone matrix.

24. The composition of claim 15, wherein said osteoinductive agent and said osteoinductive enhancer has a ratio of from about 0.01:1.0 to 100:1.0.

25. A bone repair, regeneration, maintenance, or augmentation kit for use in a bone related surgical procedures, comprising:
a bone matrix or a biocompatible matrix containing an effective amount of an osteoinductive agent; and
an osteoinductive enhancer.

26. The kit of claim 25, wherein said enhancer is provided together with the bone matrix or the biocompatible matrix as an integral product.

27. The kit of claim 25, wherein said enhancer is provided separately from the bone matrix or biocompatible matrix, capable of being stored stably for an extended period prior to use.

28. The kit of claim 25, wherein said enhancing agent is provided in a stabilizing liquid medium or as lyophilized powder to be rehydrated prior to use.

29. The kit of claim 25, wherein said bone matrix is a low activity demineralized bone matrix.

30. A bone repair, regeneration, maintenance, or augmentation method for treating a patient in need of the treatment, comprising:
applying an exogenous osteoinductive agent and an osteoinductive enhancer to a treatment site of a patient,
wherein said enhancer is capable of enhancing the in vivo activity of the osteoinductive agent.

31. The method of claim 30, wherein said enhancer is a bioflavonoid, a phytoestrogen, a mycoestrogen, a derivative thereof, or a combination thereof.

32. The method of claim 30, wherein said exogenous osteoinductive agent is a growth factor selected from the TGF-β family or a bone morphology protein.

33. The method of claim 30, wherein said osteoinductive agent and said osteoinductive enhancer are:
A: provided separately to be integrated together prior to application; or
B: provided together as a single integral product.

34. The method of claim 30, wherein said osteoinductive agent and said osteoinductive enhancer are applied separately to the patient.

35. The method of claim 30, wherein said osteoinductive agent is a growth factor selected from the TGF-β family, combinations thereof, or derivatives thereof.

36. An bone matrix formulation useful for bone repair and augmentation, comprising:
a demineralized bone matrix having embedded therein one or more osteoinductive agent; and
an effective amount of an osteoinductive enhancer.

37. The formulation of claim 36, wherein said osteoinductive enhancer is a phytoestrogen, a mycoestrogen, a derivative thereof, or a combination thereof.

38. The formulation of claim 36, wherein said osteoinductive enhancer is naringin and the demineralized bone matrix is from human bones.

39. The formulation of claim 36, wherein said osteoinductive enhancer is in the form of an additive to be added to the matrix prior to use.

40. The formulation of claim 36, wherein said osteoinductive enhancer is premixed with the demineralized bone matrix.
